(19) United States
(12) Patent Application Publication
(10) Pub. No.: US 2007/0184082 A1
(43) Pub. Date: Aug. 9, 2007

(54) Biocompatible polymeric delivery systems for sustained release of quinazolinones

(76) Inventors: Shlomo Magdassi, Jerusalem (IL); Daniel Cohn, Jerusalem (IL)

Correspondence Address: DOCKETING SPECIALIST;SULLIVAN & WORCESTER LLP, ONE POST OFFICE SQUARE, BOSTON, MA, 02109, US

(21) Appl. No.: 10/590,621

(22) PCT Filed: Feb. 25, 2004
(86) PCT No.: PCT/IL04/00189

§ 371 Date: Aug. 24, 2006

(51) Int. Cl. A61F 13/02; A61K 31/517

(52) U.S. Cl. 424/422; 424/444; 514/266.3

(57) ABSTRACT

The present invention relates to biocompatible polymeric delivery systems for controlled or sustained release of quinazolinone derivatives, including the compound halofuginone. In particular the invention relates to a polymeric delivery system comprising biocompatible polymeric beads having a two-phase core and shell structure, or polymeric films, beads or complexes that provide local sustained release of the pharmacological agent.

### FIELD OF THE INVENTION

The present invention relates to biocompatible polymeric delivery systems for controlled or sustained release of quinazolinones, including the compound halofuginone. In particular, the invention relates to polymeric delivery systems comprising biocompatible polymeric beads having a two-phase core and shell structure, or polymeric films, beads or complexes that provide sustained release of the pharmacological or bioactive agent.

### BACKGROUND OF THE INVENTION

Delivery systems and devices for controlled release of drugs are well known in the art. A variety of methods have been described in the literature, including the physiological modification of absorption or excretion, modification of the solvent, chemical modification of the drug, absorption of drug on an insoluble carrier, use of suspensions and implantation pellets. Other methods include mixing the drug with a carrier such as waxes, oils, fats, and soluble polymers, which gradually disintegrate in the physiological environment resulting in release of the drug. Much attention has been directed to the reservoir type of device, i.e., a device in which a drug is encased within a polymeric container, with or without a solvent or carrier, which allows passage of drug from the reservoir.

Another type of drug delivery device is the monolithic type in which a drug is dispersed in a polymer and from which the drug is released by degradation of the polymer and/or by passage of the drug through the polymer. The release kinetics of a drug from a polymeric delivery system are a function of the agent's molecular weight, lipid solubility, and charge as well as the characteristics of the polymer, the percent drug loading, and the characteristics of any matrix coating.

Alginate matrices have been well documented as delivery systems for water-soluble drugs. For example, U.S. Pat. No. 4,695,463 discloses an alginate based chewing gum delivery system and pharmaceutical preparations. Alginate beads have been used for controlled release of various proteins such as: tumor necrosis factor receptor in cation-alginate beads coated with polycations (Wee, S. F, Proceed. Intern. Symp. Control. Rel. Bioact. Mater., 21: 730-31, 1994); transforming growth factor encapsulated in alginate beads (Puolakkainen, P. A. et al., Gastroenterology, 107: 1319-1326, 1994); angiogenic factors entrapped in calcium-alginate beads (Downs, E. C. et al., J. of Cellular Physiology, 152: 422-429, 1992); albumin entrapped in chitosan-alginate microcapsules; (Polk, A. et al., J. Pharmaceutical Sciences, 83(2): 178-185, 1994); chitosan-calcium alginate beads coated with polymers (Okhamafe, A. O. et al., J. Microencapsul., 13(5): 497-508, 1996); hemoglobulin encapsulated with chitosan-calcium alginate beads (Huguet, M. L. et al., J. Applied Polymer Science, 51: 1427-1432, 1994), Huguet, M. L. et al., Process Biochemistry, 31: 745-751 (1996); and interleukin-2 encapsulated in alginate-chitosan microspheres (Liu, L. S. et al., Proceed. Intern. Symp. Control. Rel. Bioact. Mater, 22: 542-543, 1995).

However, the known drug delivery systems using alginate gel beads are used mainly for water-soluble drugs such as proteins or peptides. In addition, these systems suffer from lack of any sustained-release effect due to rapid release of the drug from the alginate beads (Liu, L. et al., J. Control. Rel., 43: 65-74, 1997). To avoid such rapid release, a number of the above systems attempt to use polycation polymer coatings (e.g., polylysine, chitosan) to retard the release of the protein. Alginate beads are disclosed for example in Wheatley, M. A. et al. (J. Applied Polymer Science, 43: 2123-2135, 1991) and Wee, S. F. et al. (Controlled Release Society, 22: 566-567, 1995).

Other potential drug carriers for fat-soluble drugs include liposomes and emulsions. Liposomes are defined as a structure consisting of one or more concentric lipid bilayers separated by water or aqueous buffer compartments. These hollow structures, which have an internal aqueous compartment, can be prepared with diameters ranging from 20 nm to 10 μm. They are classified according to their final size and preparation method as SUV, small unilamellar vesicles (0.5-50 nm); LUV, Large unilamellar vesicles (100 nm); REV, reverse phase evaporation vesicles (0.5 μm); and MLV, multilamellar large vesicles (2-10 μm). Depending on their composition and storage conditions, liposomes exhibit varying degrees of stability. The core micro-reservoirs of liposomes and the space between the bilayers can contain a variety of water-soluble materials (Davis S. S. & Walker I. M. 1987. Methods in Enzymology 149: 51-64; Gregorius G. (Ed) 1991. Liposomes Technology Vols. I, II, III. CRC Press, Boca Raton, Fla.; Shafer-Korting M. et al. 1989 J Am Acad Dermatol 21: 1271-1275). Liposomes can also serve as carriers for lipophilic molecules intercalated into the lipid bilayer.

Emulsions are defined as heterogeneous systems of one liquid dispersed in another in the form of droplets usually exceeding 1 μm in diameter. The two liquids are immiscible and chemically non-reactive or slowly reactive. An emulsion is a thermodynamically unstable dispersed system. Instability is a result of the system's tendency to reduce its free energy by separating the dispersed droplets into two liquid phases. Instability of an emulsion during storage is evidenced by creaming, flocculation (reversible aggregation), and/or coalescence (irreversible aggregation). Emulsions are usually used as a means of administering aqueous-insoluble drugs by dissolution of the drug within the oil phase.

Biodegradable and biocompatible polymeric films have been used in several types of medical applications in connection with implants for insertion into a patient's body. The films may be coated with or incorporate bioactive agents. Examples of such polymeric films are cited in U.S. Pat. No. 6,514,286. Polylactic acid, a copolymer of lactic acid and other aliphatic hydroxycarboxylic acid and polyester derived from aliphatic polyhydric alcohol and aliphatic polybasic acid have been known to have thermoplastic property and biodegradability. In these polymers, polylactic acid in particular is completely biodegraded in an animal body in a period of several months to one year. Polylactic acid is expected in recent years to extend its application field because the raw material L-lactic acid can be inexpensively produced in a large scale. A polylactic acid-based film is disclosed for example in U.S. Pat. No. 6,235,825.

### Halofuginone and Related Quinazolinones

Halofuginone was originally developed as an oral anti-parasitic drug in veterinary applications. U.S. Pat. No. 3,320,124, disclosed and claimed a method for treating coccidiosis with quinazolinone derivatives, one preferred embodiment being halofuginone, otherwise known as 7-bromo-6-chloro-3-[3-(3-hydroxy-2-piperidinyl)-2-oxopropyl]-4(3H)-quinazolinone. U.S. Pat. Nos. 4,824,847; 4,855,299; 4,861,758 and 5,215,993 are all related to the coccidiocidal properties of halofuginone, which has been marketed for veterinary use under the commercial name Stenorol^{R}, — as an additive in chicken feed. Consequently, a substantial body of knowledge exists regarding the chemical characterization, toxicology and pharmacokinetics of the compound (NADA document #130-951 (SBA), 1985, and The EPSA Journal 8:1-45, 2003). U.S. Pat. No. 4,340,596 further discloses the use of lactate salts of quinazolinone derivatives for the treatment of a cattle disease caused by different types of theileria.

More recently, it was disclosed in U.S. Pat. No. 5,449,678 that these quinazolinone derivatives are unexpectedly useful for the treatment of a fibrotic condition. That disclosure provides compositions of a specific fibrosis inhibitor comprising a therapeutically effective amount of a pharmaceutically active compound of the general formula (I):

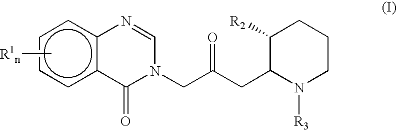

wherein: n=1-2

R₁ which may be the same or different at each occurrence is a member of the group consisting of hydrogen, halogen, nitro, benzo, lower alkyl, phenyl and lower alkoxy;

R₂ is a member of the group consisting of hydroxy, acetoxy and lower alkoxy;

R₃ is a member of the group consisting of hydrogen and lower alkenoxy-carbonyl; and pharmaceutically acceptable salts thereof.

Of this group of compounds, halofuginone has been found to be particularly effective. U.S. Pat. No. 5,449,678 discloses that these compounds are effective in the treatment of fibrotic conditions such as scleroderma and graft versus host disease (GVHD).

The ability of extremely low concentrations of halofuginone to inhibit specifically collagen type I gene expression enables broad therapeutic utility of halofuginone as a novel antifibrotic drug. Progressive fibroproliferative diseases such as liver cirrhosis (U.S. Pat. No. 6,562,829), pulmonary fibrosis (WO 98/43642) and renal fibrosis (WO 02/094178), scleroderma and a variety of other serious diseases, exhibit excessive production of connective tissue, which results in the destruction of normal tissue architecture and function.

U.S. Pat. No. 5,891,879 discloses that these quinazolinone compounds are effective in treating restenosis. Restenosis is characterized by smooth muscle cell proliferation and extracellular matrix accumulation within the lumen of affected blood vessels in response to a vascular injury (Choi et al., Arch. Surg., 130:257-261, 1995).

U.S. Pat. No. 6,159,488 discloses a stent coated with a composition for the inhibition of restenosis comprising a quinazolinone derivative including inter alia halofuginone in combination with a polymer carrier that is non-degradable.

In addition, quinazolinone containing pharmaceutical compositions including halofuginone have been disclosed and claimed as effective for treating malignancies (U.S. Pat. No. 6,028,075) as well as for prevention of neovascularization (U.S. Pat. No. 6,090,814).

Notably, halofuginone inhibits collagen synthesis by fibroblasts in vitro; however, it promotes wound healing in vivo (WO 01/17531). In addition to the fibrotic diseases with excess collagen deposition, normal wound healing involves the formation of connective tissue that consist largely of collagen fibrils. Although moderate degrees of fibrous tissue are beneficial in wound repair, fibrous material often accumulates in excessive amount and impairs the normal function of the affected tissue. Such excessive accumulation of collagen becomes an important event in scarring of the skin after burns or traumatic injury, in hypertrophic scars and in keloids.

Although the pharmacological actions of halofuginone and its therapeutic effectiveness in various diseases were extensively studied, there remains a need for improved methods of administration, particularly for long-term localized delivery of the drug in conditions amenable to treatment with this drug.

There is thus an unmet need for biocompatible polymeric delivery systems, which exhibit local sustained-release of water insoluble or poorly soluble drugs such as quinazolinones. The present invention provides novel sustained release delivery systems utilizing a polymer matrix suitable for quinazolinone derivatives.

### SUMMARY OF THE INVENTION

It is an object of the present invention to provide a biocompatible sustained release polymeric delivery system that delivers a stable therapeutic concentration of quinazolinones having the general formula (I) as defined above for extended periods ranging from a few days to a few months. Of these compounds one currently preferred compound is halofuginone.

It is another object of the present invention to provide a biocompatible polymeric delivery system for sustained release administration of a therapeutic dose of a quinazolinone having the general formula (I), to a target site in a subject, wherein the local concentration achieved at the target site is greater than that achieved when the drug is administered orally at the maximum tolerated oral dose in human patients.

The delivery systems of the present invention relate generally to a sustained release polymeric drug delivery system that is applied directly at a specific body site and permits constant and preferably local release of a quinazolinone having the general formula (I) for extended periods ranging from a few days to a few months.

The present biocompatible polymeric delivery systems are preferably suitable for the controlled release of quinazolinone derivatives having the general formula (I):

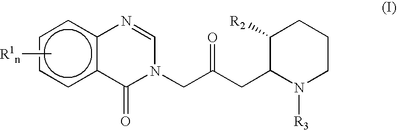

wherein: n=1-2

R₁ which may be the same or different at each occurrence is a member of the group consisting of hydrogen, halogen, nitro, benzo, lower alkyl, phenyl and lower alkoxy;

R₂ is a member of the group consisting of hydroxy, acetoxy and lower alkoxy;

R₃ is a member of the group consisting of hydrogen and lower alkenoxy-carbonyl; and pharmaceutically acceptable salts thereof. Of this group of compounds, halofuginone has been found to be particularly preferred.

As used herein, the term “lower alkyl” refers to a straight- or branched-chain alkyl group of C₁ to C₆, for example, methyl, ethyl, propyl, isopropyl, butyl, isobutyl, sec-butyl, tert-butyl, pentyl, isopentyl, hexyl, isohexyl, and the like. The term “alkenyl” refers to a group having at least one carbon-to-carbon double bond. The terms “alkoxy” and “alkenoxy” denotes —OR, wherein R is alkyl or alkenyl, respectively.

In a preferred embodiment, the delivery systems of the present invention are capable of delivering locally a therapeutic dose of halofuginone, which is higher than the maximum tolerated dose achieved when halofuginone is administered orally, without inducing the adverse symptoms associated with systemic higher doses of halofuginone. The sustained release is particularly effective since it eliminates the need for repeated doses throughout the day and avoids the fluctuations in blood levels associated with the administration of multiple daily doses.

According to one aspect, the present invention provides a polymeric sustained release delivery system for quinazolinone having the general formula (I) comprising biocompatible polymeric beads having a two-phase core and shell structure. In a preferred embodiment, the quinazolinone according to formula (I) is halofuginone, most preferably the hydrobromide or lactate salts of halofuginone. Other salts of halofuginone which may be used in the present invention are acetate and aceturate salts.

According to one embodiment the internal core comprises a water-in-oil emulsion, where halofuginone is present as a suspension in the discontinuous aqueous phase dispersed within the continuous oil phase of the emulsion. Thus, the halofuginone is entrapped within the core water-in-oil emulsion phase of the beads, while the external shell of the beads comprises a biocompatible polymeric matrix, which provides the sustained release characteristics of the system. The core and shell structured sustained release delivery system is denoted herein as “emulsion beads”.

According to another aspect, the present invention provides a polymeric sustained release delivery system for quinazolinone having the general formula (I) comprising biocompatible polymeric beads in suspension wherein the drug is substantially homogenously dispersed within the matrix of the beads. The polymeric beads in suspension are denoted herein as “Suspension beads”. In a preferred embodiment, the quinazolinone according to formula (I) is halofuginone, most preferably the hydrobromide or lactate salts of halofuginone.

The biocompatible polymeric bead matrix may be any natural or synthetic biocompatible hydrophilic polymers that are water-soluble prior to polymerization. Preferred natural biocompatible polymers to be used in the present invention are generally polysaccharides or fibrillar proteins. Polysaccharide polymers include for example alginate, dextran, cellulose and cellulose derivatives, chitosan or carrageenan. Additional polysaccharides useful according to the present invention include polyanionic polysaccharides, including dextran sulfate, chondroitin sulfate, heparan sulfate, heparin, keratan sulfate, dermatan sulfate, as well as algal polyglycan sulfates. Polymeric fibrillar proteins include for example gelatin, collagen, elastin, fibrin, and albumin. Preferred synthetic polymers to be used in the present invention are polyacrylic acid polymers, polylactic acid polymers, polycaprolactone polymers, polyglycolic acid and various copolymers thereof. Other polymers that allow the formation of beads by chemical crosslinking or heat-induced solidification may be used in the present invention.

According to yet another aspect, the present invention provides a polymeric sustained release delivery system for quinazolinone having the general formula (I) comprising polymeric complexes comprising a biocompatible negatively charged polymer conjugated through electrostatic interactions to the active compound, which is positively charged in physiological pH. In a preferred embodiment, the quinazolinone according to formula (I) is halofuginone, most preferably the hydrobromide or lactate salts of halofuginone. The polymeric complexes exhibit reduced rate of diffusion, thus providing sustained release of the conjugated active drug. Preferred negatively-charged polymers to be used in the polymeric complexes include but are not limited to polyanionic polysaccharides, including dextran sulfate, chondroitin sulfate, heparan sulfate, heparin, keratan sulfate, dermatan sulfate, as well as algal polyglycan sulfates.

According to one currently preferred embodiment the anionic polysaccharide of the polymeric complexes according to the invention is an alginate polysaccharide, which is negatively charged at physiological pH.

In yet another aspect, the present invention provides a polymeric sustained release delivery system for quinazolinone having the general formula (I) comprising biocompatible polymeric films in which the active drug agent is retained within the matrix of the film. In a preferred embodiment, the quinazolinone according to formula (I) is halofuginone, most preferably the hydrobromide or lactate salts of halofuginone. The polymeric films exhibit a homogenous distribution of the active drug within the polymeric matrix and sustained release of halofuginone for prolonged periods. Unexpectedly, the polymeric film comprising halofuginone entrapped therein exhibit consistent local delivery of a predetermined therapeutic concentration of halofuginone for extended periods. Furthermore, the polymeric films of the present invention may be prepared so that the rate of bio degradation is controlled. Such control can be achieved by controlling the composition of the polymeric film as well as by the morphology of the polymer. According to one embodiment, the concentration of the active drug may very from about 0.1 to about 20% per polymer weight, preferably from about 1 to about 10% w/w.

The polymeric delivery system of the present invention permit sustained release of a therapeutic dose of a quinazolinone

According to another embodiment, the polymeric films of the present invention may also be used as a coating layer of a suitable matrix, a device or an implant. A coating according to the present invention is useful for all materials which are directly introduced into the bloodstream, e.g. for vascular prostheses, stents, artificial heart valves, as well as for implants which are in contact with tissue, e.g. cardiac pacemakers or defibrillators and for implants which are in contact with body fluids, e.g. bile duct drains, catheters for draining urea and cerebrospinal fluid, and endotracheal resuscitation tubes, and even for implants used in orthopedic surgery including bone implants, cartilage implants, artificial joints and the like.

In one aspect, the polymeric drug delivery system of the invention may be implanted to a target site or cavity within a subject as part of an implanted system preferably via a minimally invasive surgical procedure. According to certain embodiments, preferred locations of the implanted delivery system are subcutaneous, or within a body cavity such as a cavity formed following the removal of a tissue during surgery or within any natural body cavity. Such locations may be for example in the brain, kidney capsule, bladder, uterus, vagina, joints, lungs, and peritoneum.

The delivery system of the invention may be applied topically to the desired site for treatment of an intact organism. Suitable sites for topical application of the system include but are not limited to: the skin for dermal administration or transdermal administration, mucosal surfaces including intranasal administration or buccal administration; topical delivery to the lungs by inhalation in the form of aerosols. According to alternative embodiments, the delivery system is administered to the desired location as part of an implanted system or may be positioned directly at the desired site, preferably via a minimally invasive surgical procedure.

The sustained release polymeric delivery system of the present invention exhibits significant advantages over the existing art. Unexpectedly, the beads delivery system permits continuous release of halofuginone for prolonged periods and avoids the high initial burst release of the drug as is associated with certain other polymeric delivery systems. Furthermore, the film delivery system of the present invention exhibits negligible cleavage of the polymer backbone or mass loss over a period up to several months. Thus, a predetermined rate of release of halofuginone is possible for extended periods ranging from a few days to a few months. Moreover, the polymeric delivery system of the present invention may be structured into an article of a desired shape and size, enabling its application to or at different body locations. The delivery system of the present invention is suitable for incorporating any quinazolinone derivative of formula (I) while preserving its bioactivity upon exposure to the encapsulation polymer.

While the drug delivery systems of the present invention is referred to throughout the specification and claims as “beads” or “films”, it is to be understood that these terms are intended to be construed in a non-limitative fashion, and do not imply any requisite geometry, specific shape or size of the product. It is noted that the diameter of the beads may vary from several microns to several hundred of microns. Similarly, the dimensions and shape of the films may vary, depending on the target site of application.

In yet another aspect, the present invention provides methods of preparing the biocompatible polymeric delivery systems of the present invention. In one embodiment, a method of preparing core-and-shell-structured polymeric beads comprising the quinazolinone derivative of formula (I) is disclosed. The method comprising: mixing an aqueous suspension comprising the quinazolinone derivative of formula (I) in an oily phase to form a water-in-oil emulsion; homogenizing the mixture; applying a polymeric shell solution with a cross linking agent to the homogenized mixture, and forming core-and-shell-structured polymeric beads. Of this group of compounds, halofuginone has been found to be particularly preferred.

In another aspect, a method of preparing polymeric films comprising the quinazolinone derivative of formula (I) homogenously entrapped therein is disclosed. The method comprising dissolving the active drug in an organic solvent to form a drug solution; mixing a polymer in suitable solvent to form a polymeric solution; mixing the drug solution with the polymeric solution, and evaporating the polymer solvent to form the polymeric films comprising the quinazolinone derivative of formula (I) homogenously entrapped therein. Of this group of compounds, halofuginone has been found to be particularly preferred.

In another aspect, a method of preparing polymeric complexes comprising the quinazolinone derivative of formula (I) is disclosed. The method comprising dissolving the quinazolinone derivative of formula (I) in an organic solvent to form a drug solution; mixing the polymer in suitable solvent to form a polymeric solution; mixing the drug solution with the polymeric solution for sufficient time to form polymeric complexes; and precipitating the polymeric complexes. Of this group of compounds, halofuginone has been found to be particularly preferred.

In another aspect, a method of preparing Suspension beads comprising the quinazolinone derivative of formula (I) is disclosed. The method comprising suspending the quinazolinone derivative of formula (I) in an aqueous solution to form a drug suspension; mixing the polymer in suitable solvent to form a polymeric solution; mixing the polymeric solution with a cross linking agent and the drug suspension; and forming polymeric beads comprising said quinazolinone derivative of formula (I). Of this group of compounds, halofuginone has been found to be particularly preferred, most preferably hydrobromide or lactate salts of halofuginone.

In yet another aspect, the present invention provides a method of delivering a stable therapeutic concentration of the quinazolinone derivative of formula (I) for extended periods comprising: administrating to a mammal in need the biocompatible polymeric delivery system of the present invention comprising the active drug, wherein the delivery system continuously delivers a stable therapeutic concentration of the drug for extended periods. Preferably, the delivery system continuously delivers the drug to a specific location in the body. Of this group of compounds, halofuginone has been found to be particularly preferred.

In yet another aspect, the present invention provides a method of treating a disease in which inhibition of angiogenesis, prevention of tumor growth, prevention of smooth muscle cells proliferation or blocking of extracellular matrix deposition (fibrosis) is required, comprising administering to a subject in need the biocompatible polymeric delivery system of the present invention, wherein the delivery system comprising halofuginone entrapped therein, said delivery system continuously delivers a stable therapeutic concentration of halofuginone for extended periods, thereby treating the disease.

These and further embodiments will be apparent from the detailed description and examples that follow.

### BRIEF DESCRIPTION OF THE DRAWINGS

FIG. 1 shows the percentage of halofuginone released over time from alginate beads and polymeric complexes of alginate and poly acrylic acid at 37° C.

FIG. 2 shows the consistent drug release from the alginate Emulsion beads over time at 37° C.

FIGS. 3-6 show the release of halofuginone from the Emulsion beads, the Suspension beads and the polymeric complexes, expressed as the concentration of drug (mg/ml) in the external PBS buffer at 37° C.

FIGS. 7-8 show the percent of halofuginone released over time from alginate beads and polymeric complexes of alginate at room temperature.

FIGS. 9-12 demonstrate the release of halofuginone from the Emulsion beads, the Suspension beads and the polymeric complexes, expressed as the concentration of drug (mg/ml) in the external PBS buffer at room temperature.

FIG. 13 demonstrates the mechanical strength of the halofuginone-polycaprolactone polymeric films.

FIG. 14 shows the calibration curve for determining the concentration of halofuginone by UV spectroscopy.

FIG. 15 demonstrates the total amount of halofuginone (mg) released from the polycaprolactone film at 37° C.

FIG. 16 demonstrates the percentage halofuginone released (%) from the polycaprolactone film at 37° C.

FIG. 17 shows the effect of the degradation of polycaprolactone film on its morphology during its immersion in PBS as studied by the DSC thermogram.

FIG. 18 shows the DSC thermogram of polycaprolactone film containing 10% halofuginone.

FIG. 19 shows the DSC thermogram of polycaprolactone film containing 10% halofuginone after 43 days.

FIG. 20 shows the drug release (mg) from a polyethylene terephthlate film coated with halofuginone-containing polycaprolactone film.

FIG. 21 shows the percent drug release from polyethylene terephthlate film coated with halofuginone-containing polycaprolactone film.

FIG. 22 shows the DSC thermogram of polyethylene terephthlate film coated with polycaprolactone film.

FIG. 23 shows the DSC thermogram of polyethylene terephthlate film coated with 10% halofuginone-containing polycaprolactone film.

FIG. 24 shows DSC thermogram of polyethylene terephthlate film coated with 10% halofuginone-containing polycaprolactone after 15 days.

FIG. 25 shows the plasma concentration of halofuginone measured in patients receiving a single oral dosing of 2 mg of halofuginone.

FIG. 26 shows the amount of halofuginone excreted to the urine examined in 3 patients who were administered with a single oral dose of 2 mg halofuginone.

### DETAILED DESCRIPTION OF THE INVENTION

The present invention relates to biocompatible polymeric delivery systems that permit controlled release of the quinazolinone derivative of formula (I). In a preferred embodiment, the quinazolinone according to formula (I) is halofuginone, most preferably the hydrobromide or lactate salts of halofuginone. The polymeric delivery systems deliver stable amounts of the active drug for prolonged time periods, preferably within specific location in the body. Variations in the volume of the polymeric matrix provide flexibility in the amount of drug released per time period, and the total duration of drug release. Importantly, the present systems eliminate the need for multiple doses administration of the pharmacological agent to the subject in need thereof and the fluctuations in drug concentration associated therewith.

In a preferred embodiment, the delivery systems of the present invention are capable of delivering locally a therapeutic dose of halofuginone which is higher than that achieved by oral administration of the maximum tolerated dose. Thus, for example, it is possible to administer halofuginone locally and achieve a therapeutic level higher than that achieved by oral administration of 1 mg/day, which is the maximum tolerated dose of halofuginone with no adverse effects observed in humans when administered orally.

Importantly, the delivery systems of the present invention may avoid or reduce the adverse effects observed with the oral or systemic administration of the drug. Significantly, the use of the sustained release at a target site avoids the need for multiple daily doses and the resultant fluctuations in serum levels associated therewith.

Halofuginone is a quinazolinone derivative which was initially used as a coccidiocidal drug but was further discovered to be effective in treating fibrotic diseases, as well as for treatment of restenosis, mesangial cells proliferation, and angiogenesis-dependent diseases (disclosed for example in U.S. Pat. Nos. 6,159,488, 5,998,422 6,090,814 and 6,028,075). As disclosed hereinabove, in a preferred embodiment the present polymeric delivery system comprises halofuginone as the active drug. The halofuginone-polymeric beads or halofuginone-polymeric films of the present invention exhibit prolonged release of halofuginone over a period of several months.

As disclosed herein, in one embodiment the present drug release systems comprise biocompatible Emulsion and Suspension beads. It is noted that the biocompatible polymeric bead matrix may be any natural or synthetic biocompatible hydrophilic polymers. Hydrophilic polymers including alginates and derivatives thereof can be obtained from various commercial, natural or synthetic sources well known in the art. As used herein, the term hydrophilic polymer refers to water-soluble polymers or polymers having affinity for absorbing water. Hydrophilic polymers are well known to one skilled in the art. These include but are not limited to polyanions, including anionic polysaccharides such as alginate, carboxymethyl amylose, polyacrylic acid salts, polymethacrylic acid salts, ethylene maleic anhydride copolymer (half ester), carboxymethyl cellulose, dextran sulfate, heparin, carboxymethyl dextran, carboxy cellulose, 2,3-dicarboxycellulose, tricarboxycellulose, carboxy gum arabic, carboxy carrageenan, pectin, carboxy pectin, carboxy tragacanth gum, carboxy xanthan gum, pentosan polysulfate, carboxy starch, carboxymethyl chitin/chitosan, curdlan, inositol hexasulfate, β-cyclodextrin sulfate, hyaluronic acid, chondroitin-6-sulfate, dermatan sulfate, heparin sulfate, carboxymethyl starch, carrageenan, polygalacturonate, carboxy guar gum, polyphosphate, polyaldehydo-carbonic acid, poly-1-hydroxy-1-sulfonate-propen-2, copolystyrene maleic acid, agarose, mesoglycan, sulfopropylated polyvinyl alcohols, cellulose sulfate, protamine sulfate, phospho guar gum, polyglutamic acid, polyaspartic acid, polyamino acids, derivatives or combinations thereof. One skilled in the art will appreciate other various hydrophilic polymers that are within the scope of the present invention.

As disclosed hereinabove, in one embodiment the present drug release systems comprise a biocompatible, preferably non-biodegradable polymeric film. As used herein “non-biodegradable” refers to polymers which degrade in a time scale which is not relevant to the present invention, i.e. the degradation time scale is significantly longer compared to the drug treatment time scale. For example, Poly(ε-Caprolactone) (PCL) degrades very slowly, in a time scale of about 2 years and, therefore, it can be seen as being non-biodegradable in the time scale relevant to the present invention. Preferred polymers suitable for preparing the drug-loaded films include polymers, which exhibit sufficient mechanical strength after polymerization following the incorporation of the active drug to the polymerization solution. Suitable polymers are for example Poly(ε-Caprolactone), (PCL), Poly(L-Lactide) (PLLA) and block copolymers of these polymers

The polymeric films of the present invention may also be used as a coating layer of a suitable matrix. Various artificial materials are introduced into the human body as a short-term or relatively long-term implant for diagnosis and treatment (catheters, probes, sensors, stents, artificial heart valves, endotracheal tubes, bone implants, cartilage implants, artificial joints, and the like). The selection of the material for these implants depends on the stability and geometry required to insure a certain function of the implant. In order to meet these functional demands, it is often not possible to pay sufficient regard to the fact of whether these materials are biocompatible. Therefore, it is useful to improve the materials from which these implants are made by coatings which are compatible with blood and tissue. Desired attributes of these coatings are that they activate the coagulation system only to a minor degree, and that they cause few endogenous defense reactions thus reducing the deposit of thrombi and biofilm on the implant surface. A coating is useful for all materials which are directly introduced into the bloodstream, e.g. for vascular prostheses, stents, artificial heart valves, as well as for implants which are in contact with tissue, e.g. cardiac pacemakers or defibrillators and for implants which are in contact with body fluids, e.g. bile duct drains, catheters for draining urea and cerebrospinal fluid, and endotracheal resuscitation tubes. The blood compatibility of implants is influenced decisively by their surface properties. In order to avoid the formation of thrombi it is advantageous that the implant exhibit relative smoothness is necessary to prevent the deposit and destruction of corpuscular components of the blood and activation of the coagulation system. Coating the matrix may be performed by any suitable method as is known in the art. According to one embodiment, an implant such as a stent is dipped in a solution of the polymer. The solvent type, the polymer concentration and the rate of evaporation may vary according to the intended use, particularly according to the preferred pattern of release.

Drug-loaded films are advantageously fabricated by a solvent casting technique. The polymer is first dissolved in an organic solvent, preferably a low boiling solvent such as tetrahydrofuran (THF) to facilitate eventual removal of the solvent by evaporation. The concentration of the polymer solution advantageously ranges from about 0.1% wt to about 20% wt, preferably five to twenty % wt.

The drug to be embedded in the film is first dissolved prior to its dispersion into the polymer solution. As shown in the following examples, the dissolution of the halofuginone prior to its incorporation into the polymer solution reduces the brittleness of the films. Reasonably reproducible release kinetics (i.e., near constant delivery) are obtained with commercially available drug particles which have been micronized. Preferred concentration of the drug may vary from about 0.1 to about 20% per polymer weight, more preferably, between 1-10% w/w.

The polymer solution with drug is then cast into a mold of the desired shape and size. After slow evaporation of the solvent, the drug molecules or drug particles are embedded in the polymer matrix. The casting is typically done at low temperatures to prevent sedimentation of the drug particles during the solvent evaporation. Typically the polymer solution with drug is poured into a mold that has been cooled to a temperature below the melting point of the solvent.

The preferred range of the active ingredient in the coating may constitute up to 10% w/w of the drug in the polymeric matrix. The drug/polymer mixture is homogeneous and the drug is dispersed homogenously throughout the polymeric matrix. The thickness of the polymeric film is advantageously a few hundred of microns, preferably 1-2 mm.

According to one embodiment, the delivery system of the invention is implanted directly to the site of action, preferably via a minimally invasive surgical procedure. For example, the system of the invention may be implanted subcutaneously, using procedures known to those skilled in the art. When beads are used as the delivery system, they may be administered subcutaneously by injection using appropriate syringes. In another embodiment, the system of the invention may be implanted in any body cavity such as for example via laparoscopy, or endoscopy. In another embodiment, the system of the invention may be implanted in any body cavity such as for example in the uterus, brain, kidney capsule, bladder, vagina, joints, lungs, and peritoneum. In yet another embodiment, the system of the invention may be implanted in a cavity formed during a surgical procedure, such as but not limited to surgery for the removal of a malignant tissue.

In another embodiment, the delivery system of the invention may be applied topically in a target site of an intact organism. Preferred targets for topical application of the system are for example: the skin using transdermal administration, intranasal administration and topical delivery to the lungs as aerosols. For transdermal administration it is desirable that the beads will be dispersed with oils to provide an oily suspension, emulsion, cream or gel.

One skilled in the art will be able to ascertain effective dosages of halofuginone to be administered via the delivery systems of the present invention by administration and observing the desired therapeutic effect. The dosage of the sustained-release preparation is the amount necessary to achieve the effective concentration of halofuginone in vivo, for a given period of time. The dosage and the preferred administration frequency of the sustained-release preparations vary with the desired duration of the release, the target disease, desired administration frequency, the subject animal species and other factors. Preferably, the total amount of halofuginone to be administered via the delivery systems of the present invention may be between about 0.1 mg/day and about 10 mg/day.

As disclosed in the Examples, a preferred delivery system comprises halofuginone as the active drug. In this particular case, the delivery system of the invention can be used in treating fibrotic diseases, restenosis, glomerulosclerosis, cancer and other angiogenesis-dependent diseases. The delivery system comprising halofuginone may be preferably used in treating diseases in which inhibition of tumor progression by cell cycle arrest, cell invasiveness or inhibiting angiogenesis is required, or in treating diseases in which blocking of extracellular matrix deposition is required. Clinical conditions and disorders associated with primary or secondary fibrosis, such as systemic sclerosis, graft-versus-host disease (GVHD), pulmonary and hepatic fibrosis and a large variety of autoimmune disorders are distinguished by excessive production of connective tissue, which results in the destruction of normal tissue architecture and function. These diseases can be interpreted in terms of perturbations in cellular functions, a major manifestation of which is excessive collagen deposition.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

Experimental Procedures

Both Emulsion and Suspension beads experiments were conducted with micronized halofuginone (HF HBr), batch H001. A water-in-oil emulsion was prepared, in which the 20% wt internal phase contained 50 mg HF HBr/ml and the oil was sunflower oil. The emulsion was prepared by adding the aqueous HF solution (containing 50 mg/ml HF HBr, 0.3% wt Tween 80) into the oil which contains 2.7% wt Span 80, and homogenizing by an Ultra Turrax homogenizer (2 min at 13,000 rpm and 10 min at 16,000 rpm). Beads were formed by a core-shell double nozzle Innotek (500 and 400 microns), flow rate of the core material 90 (instrument scale) pressure (shell) 0.6 Atm. The shell solution was 2.5% sodium alginate (FMC LF10/60) and 2.5% silica in aqueous solution (Theoretical Shell/core weight ratio 15:1 by volume).

The crosslinking solution was 100 mM CaCl₂, or 100 mM NaCl+100 mM CaCl₂. The purpose of the crosslinking solution is to provide the insoluble polymeric coating. The properties of the polymeric shell depend on various parameters, such as the NaCl/CaCl₂ ratio.

For the release experiments, 300 mg beads were suspended in 1 ml PBS buffer, and put into a dialysis tube, while the tube immersed in 10 ml PBS. Therefore, the maximal concentration of HF, which can be released, is 0.36 mg/ml based on the total amount of the drug and the volumes during the dialysis experiment. For all experiments, the concentration measurements were performed by a UV-spectrophotometer, using a calibration curve of HF PBS solution. The dialysis was performed while shaking, at 37° C. at 5 strokes/min. HF in emulsion or in suspension (“drug emulsion” and “drug suspension”, respectively) served as a control. The external buffer was completely replaced after each measurement.

### Example 1

### Extended Release of Halofuginone (HF) using Alginate Beads and Halofuginone-polymeric Complexes

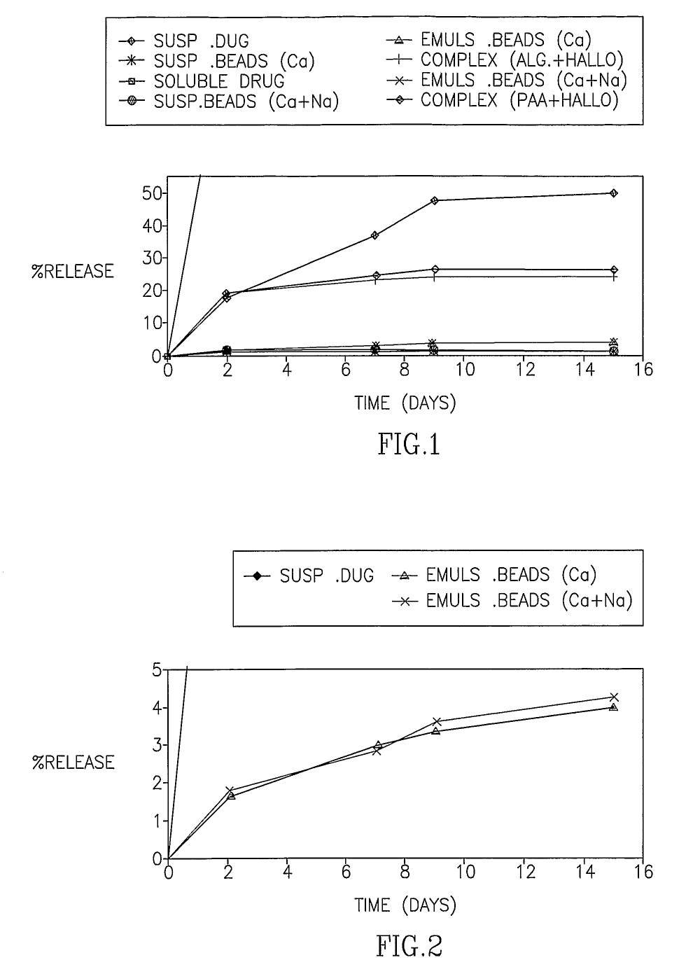

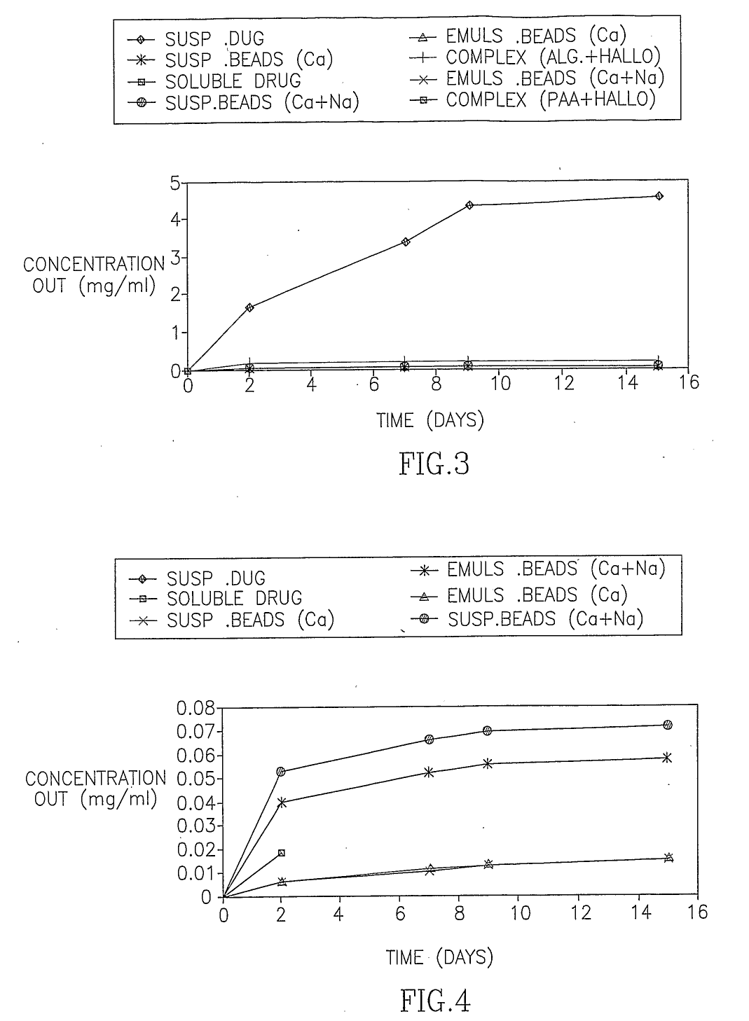

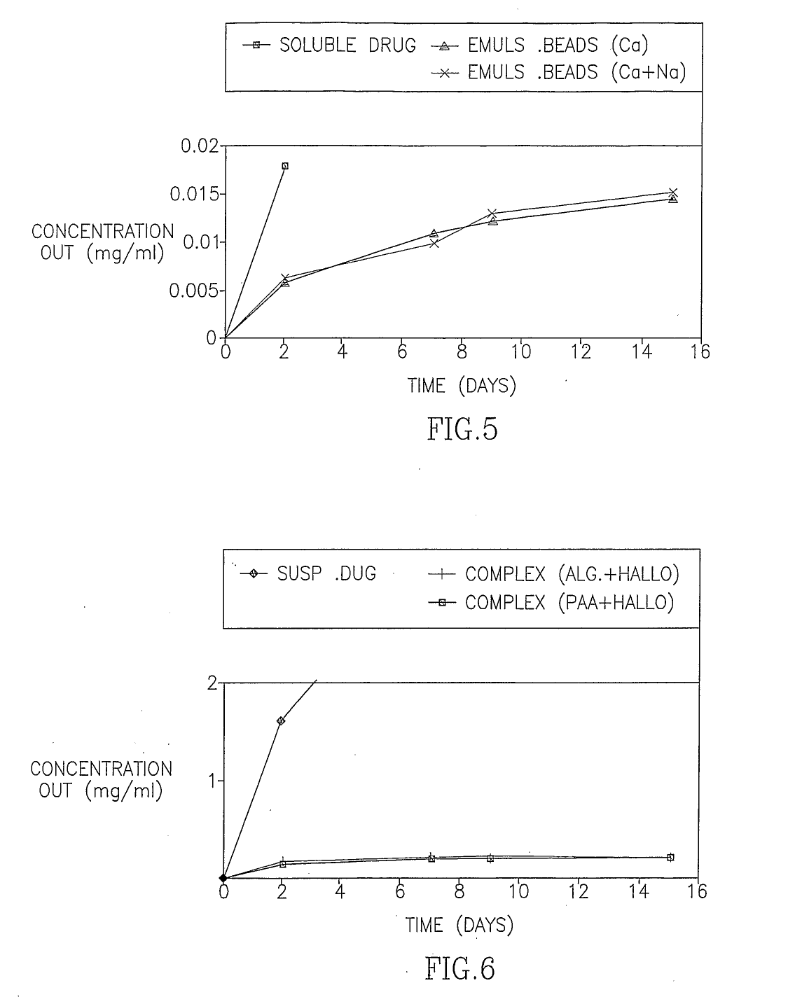

In the first set of experiments, the release of HF from the Emulsion beads, the Suspension beads and the polymeric complexes was examined in 37° C. The release pattern is presented both as the percentage of drug released of the total expected drug release and as the actual measured concentration. FIG. 1 demonstrates the cumulative percentage of HF released over time from alginate beads and polymeric complexes of alginate and poly acrylic acid (PAA). FIG. 2 is an enlargement of FIG. 1 demonstrating the consistent drug release from the Emulsion beads over time. FIGS. 3-6 demonstrate the release of HF from the Emulsion beads, the Suspension beads and the polymeric complexes, expressed as the cumulative concentration of drug (mg/ml) in the external PBS buffer.

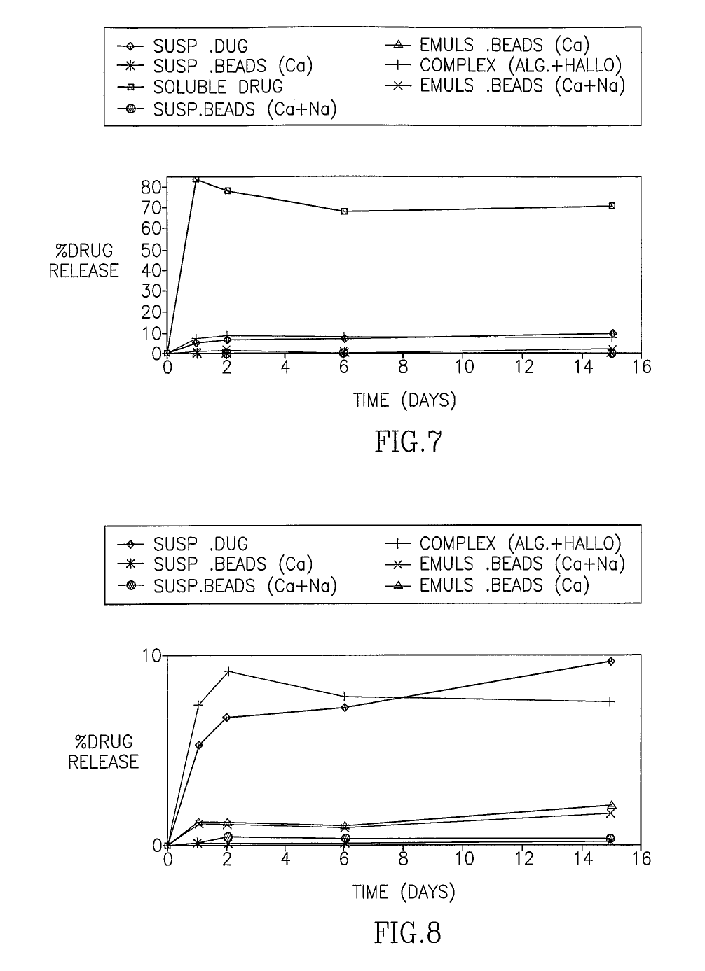

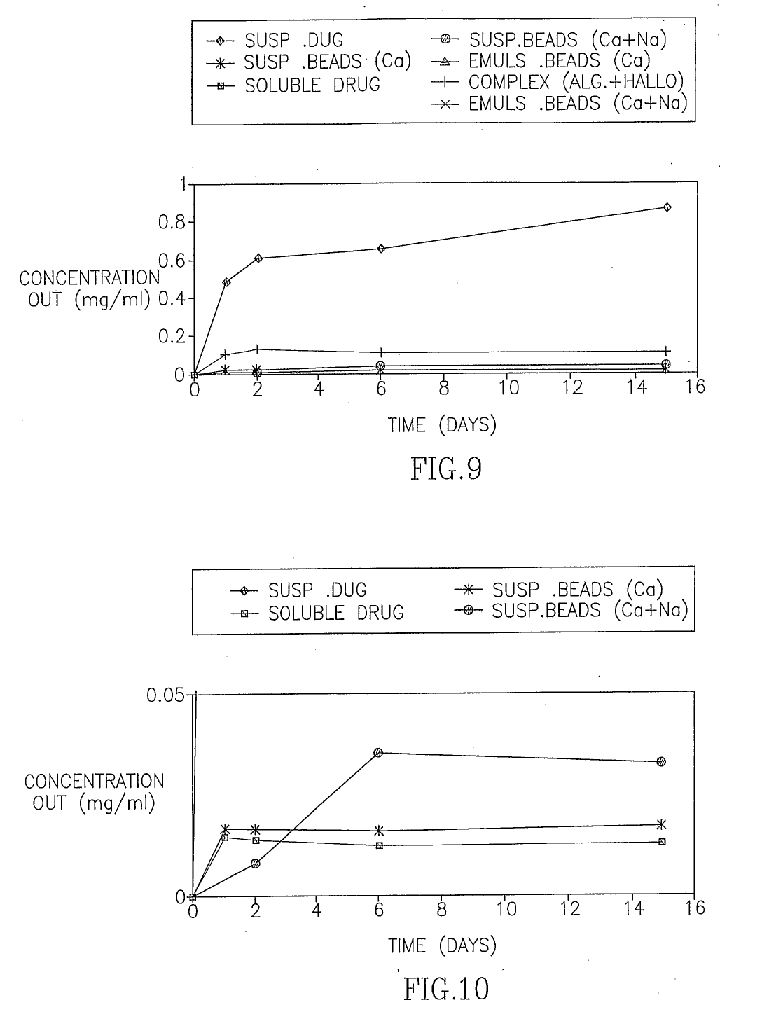

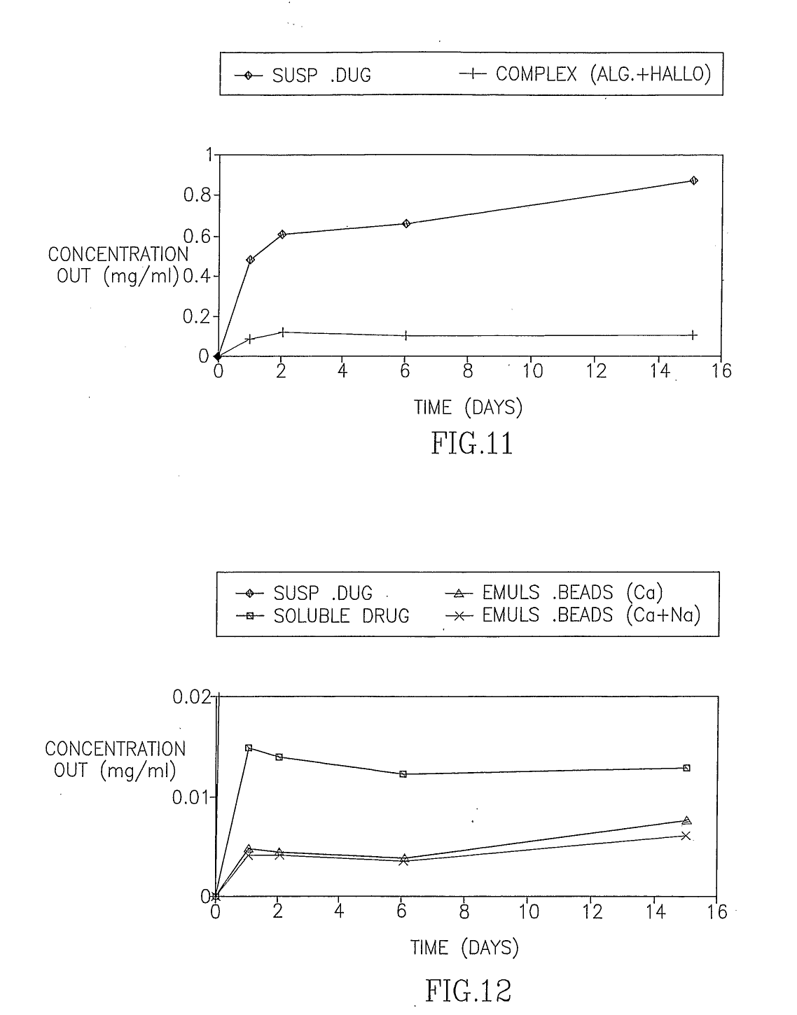

In the second set of experiments, the release of HF from the Emulsion beads, the Suspension beads and the polymeric complexes was examined at room temperature. FIGS. 7 and 8 demonstrate the cumulative percentage of HF released over time from alginate beads and polymeric complexes of alginate. FIGS. 9-12 demonstrate the release of HF from the Emulsion beads, the Suspension beads and the polymeric complexes, expressed as the cumulative concentration of drug (mg/ml) in the external PBS buffer.

As demonstrated in FIGS. 1-12, it is possible to use the Emulsion beads and the Suspension beads as a delivery system for HF. Furthermore, the drug release from the beads is much slower as compared to the dialysis rate of HF in solution or in suspension.

### Example 2

### Extended Release of Halofuginone using Halofuginone-polymeric Films

The following experiments were conducted in order to determine the feasibility of delivering halofuginone in a controlled fashion from biocompatible polymeric films and polymeric-coated articles. The polymers tested were (a) polycaprolactone (PCL) and (b) poly(l)lactic acid (PLA). These two polymers combine enhanced hydrophobicity and high crystallinity and, therefore, their rate of degradation is extremely slow. These polymers have been used extensively in the biomedical field.

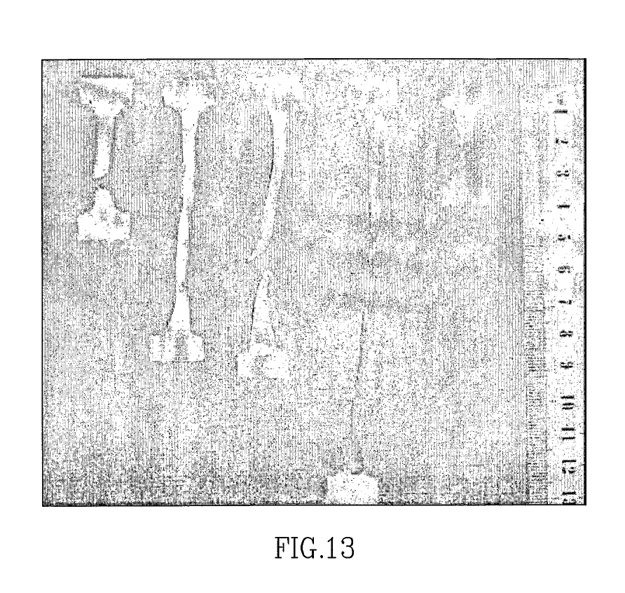

Table 1 below summarizes the mechanical data obtained with the halofuginone-PCL films. FIG. 13 demonstrates the mechanical strength of the halofuginone-PCL polymeric films. It is apparent from both the mechanical data of Table 1 and FIG. 13 that halofuginone microparticles dramatically weakened the film as well as sharply increased its brittleness. However, dissolution of the drug in ethanol:water mixture prior to its incorporation into PCL's THF solution largely reduced this detrimental phenomenon, resulting in a halofuginone-containing PCL film that retained most of the mechanical features of the film without the drug.

TABLE 1

| | Stress at | Strain at | |
|---|---|---|---|
| | break | break | Modulus |
| Sample | [MPa] | [%] | [GPa] |
| PCL | 33 | 373 | 1.3 |
| PCL + 10% HF dispersion in THF | 6 | 59 | 0.4 |
| all the solutions are in THF | | | |
| PCL + 5% HF in EtOH/H₂O | 20 | 320 | 0.7 |
| PCL + 10% HF in EtOH/H₂O | 20 | 302 | 0.6 |

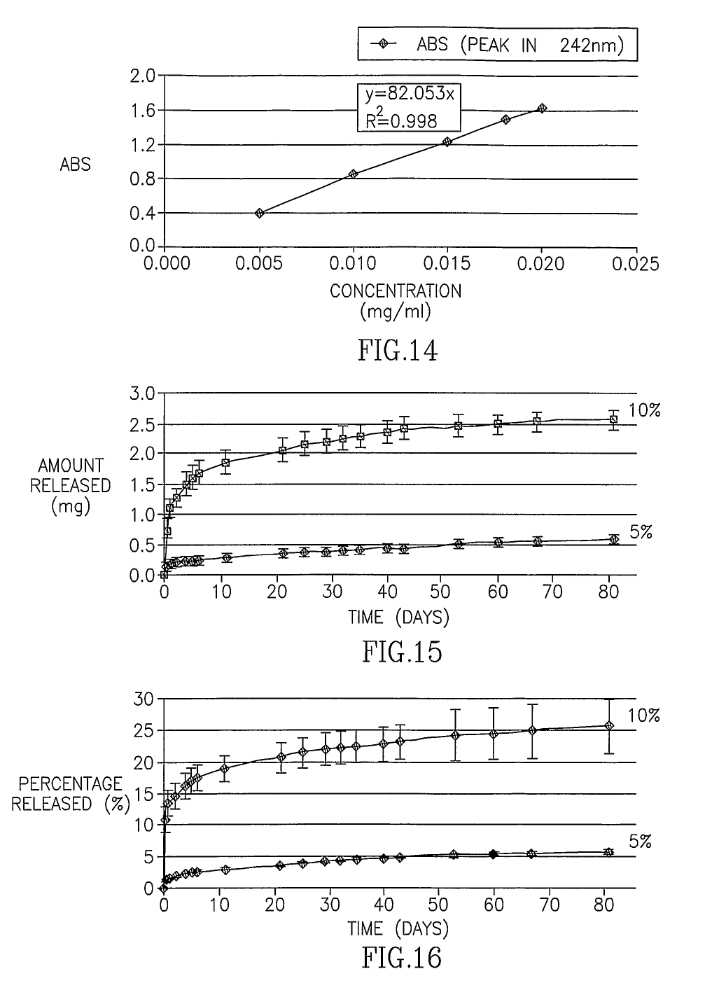

The next step was to study the release of halofuginone from the PCL films into a PBS buffer solution (pH=7.4; 0.1 M). The presence of the drug in the solution was determined by UV spectroscopy, focusing on the maximum peak at 242 nm. Table 2 presents the concentration versus absorbance (ABS) data, while FIG. 14 shows the halofuginone calibration curve.

Dissolution conditions: A film weight of 0.163±0.03 gram was put into 28 ml glass vials, with 25 ml of PBS. Halofuginone release was examined with shaking at 5 strokes/min, at 37° C. The external buffer was completely replaced after each measurement. At each time point three samples were analyzed.

TABLE 2

| Concentration | Absorbance |
|---|---|
| mg/ml | at 242 nm |
| 0.005 | 0.426 |
| 0.010 | 0.850 |
| 0.015 | 1.217 |
| 0.018 | 1.490 |
| 0.020 | 1.621 |

Films containing 5 and 10% w/w of HF were prepared following the pre-dissolution step described above and the release of HF at 37° C. was followed for 80 days. Data are presented as a mean amount of drug released (mg) as well as percentage of drug released compare to the initial amount of drug present in the film (%). FIG. 15 demonstrates the cumulative amount of halofuginone (mg) released from the PCL film. FIG. 16 demonstrates the cumulative percentage of halofuginone released (%) from the PCL film.

Two different release stages are apparent from the data presented in FIGS. 15 and 16. There is a burst effect during an initial short period of approximately 24 hours, due to the release of drug present on the surface of the PCL film, followed by a long period characterized by a very slow release kinetics. Higher HF concentrations within the film resulted in a more pronounced burst effect. Once the burst effect ended, the hydrophobicity and crystallinity of PCL affected the thermodynamics and kinetics of the process, respectively, resulting in the slow release rate measured. A crude extrapolation from the data points shown, based on the average amount of drug delivered per day starting the fifth day, indicates that films initially containing 10% and 5% drug, will deliver halofuginone for a total of 120 and 230 days, respectively.

In order to assess the extent of degradation of the polymer during the release period, PCL's molecular weight was determined by Gel Permeation Chromatography (GPC). Even though GPC data tend to show considerable fluctuations, the molecular weight of PCL samples do show an increase after 43 days in PBS at 37° C. (see Table 3).

TABLE 3

| Sample | Mn | Mw | Pd |
|---|---|---|---|
| PCL | 83,758 | 120,271 | 1.4 |
| PCL + 5% HF | 100,320 | 140,577 | 1.4 |
| PCL + 5% HF after 43 days in PBS | 122,392 | 162,923 | 1.3 |
| PCL + 10% HF | 111,637 | 151,293 | 1.4 |
| PCL + 10% HF after 43 days in PBS | 127,535 | 165,622 | 1.3 |

Mn—The number average molecular weight, Mn is the simple average of total mass of the chains divided by the number of chains.
Mw—The weight average molecular weight, Mw is the sum of the square molecular weight divided by the sum of the molecular weight of all the molecules present
Pd—Polydispersity, molecular weight distribution

This phenomenon can be attributed to the removal of low molecular weight fragments from the matrix, leaving behind a matrix with a higher average molecular weight. Furthermore, these fragments would result also in an increase in the crystallinity of the polymer, since it is mainly the amorphous material that degrades initially.

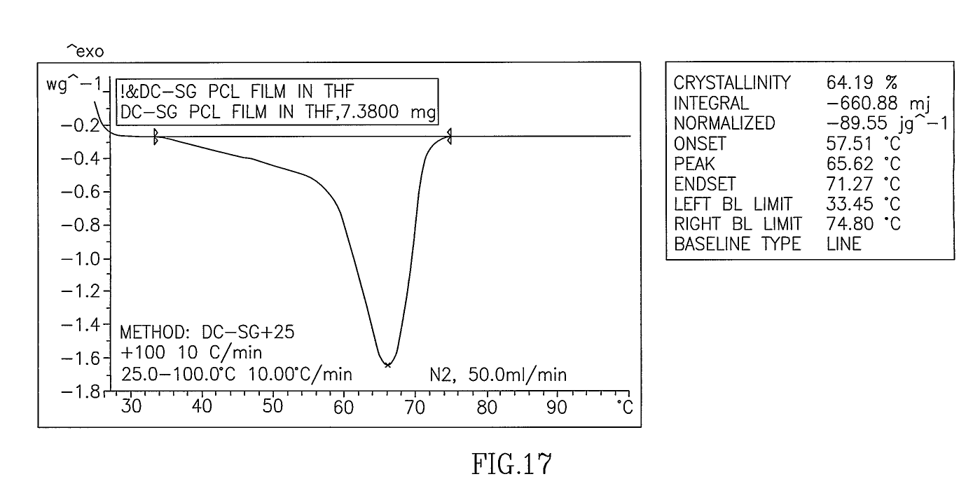

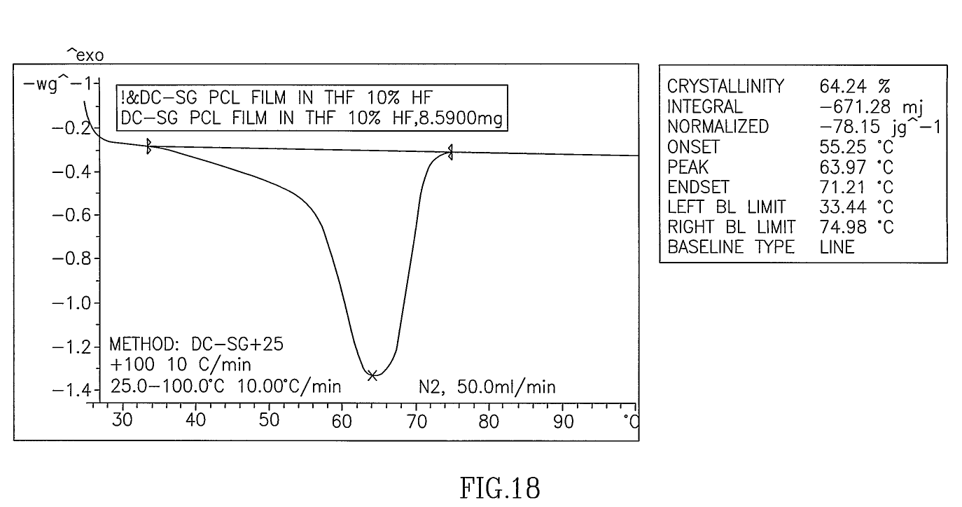

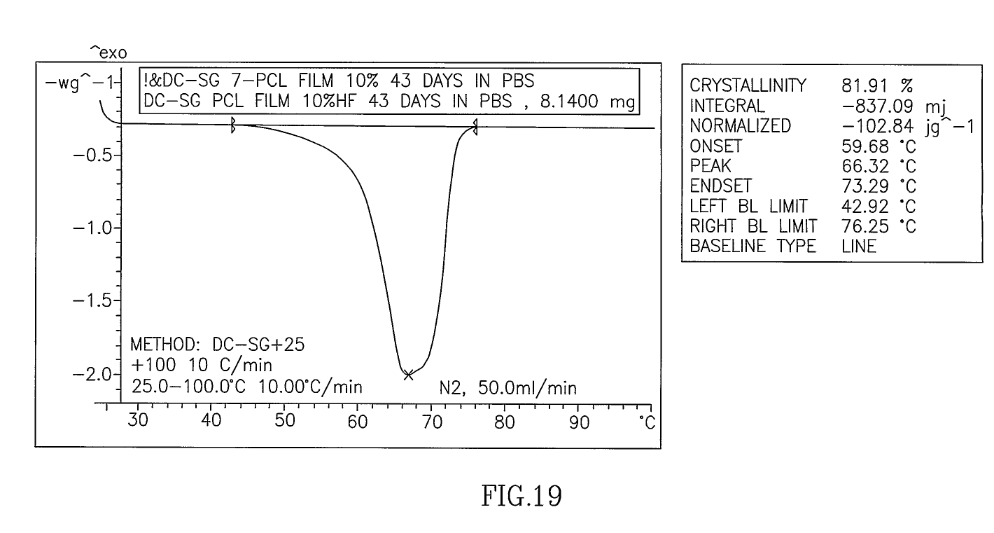

The effect of the degradation of PCL on its morphology during its immersion in PBS, was studied by Differential Scanning Calorimetry (DSC). The thermograms shown in FIGS. 17-19 and the data summarized in Table 4, clearly show that PCL's degree of crystallinity increased after 43 days in PBS at 37° C. These findings are in full accordance with the increase in molecular weight revealed by the GPC data and with the prediction of an increase of crystallinity following the removal of amorphous material from the polymeric matrix. These data revealed that even though some degradation has taken place, it is clear that it is far from representing the limiting factor affecting the length of release period.

TABLE 4

| Sample | PCL crystallinity (%) |
|---|---|
| PCL | 64 |
| PCL + 10% HF | 62 |
| PCL + 10% HF after 43 days in PBS | 82 |

### Example 3

### Extended Release of Halofuginone from Metal and Polymeric Carriers Coated with Halofuginone-PCL Films

Metal and polymeric samples were coated with halofuginone-containing PCL films. Polyethylene terephthlate (PET) is one of the most important biomedical polymers presently used in the cardiovascular area, where they represent the largest family of vascular grafts.

PET films were coated by dipping the films in a PCL 10% w/w THF solution. After dipping for 2 minutes, the solvent was evaporated and a 50 μm to 100 μm coating layer was formed with a weight increase of approximately 50%. Halofuginone-containing PCL coatings were prepared by dipping PET films into 5 and 10% w/w halofuginone dispersion in THF.

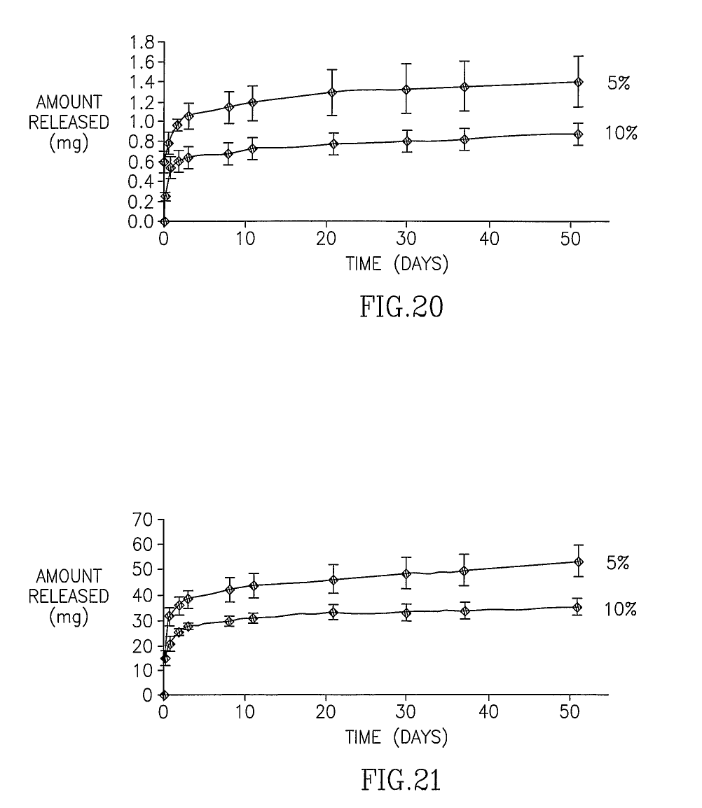

The release of halofuginone from the PET/PCL bi-layered films into a PBS buffer solution (pH=7.4 0.1M, 37° C.) was studied. The presence of the drug in solution was determined as previously, by UV spectroscopy, focusing on the maximum peak at 242 nm. As apparent from the data presented in FIGS. 20 and 21, the release rate of halofuginone from the coated system is much higher than the one measured previously for the PCL films.

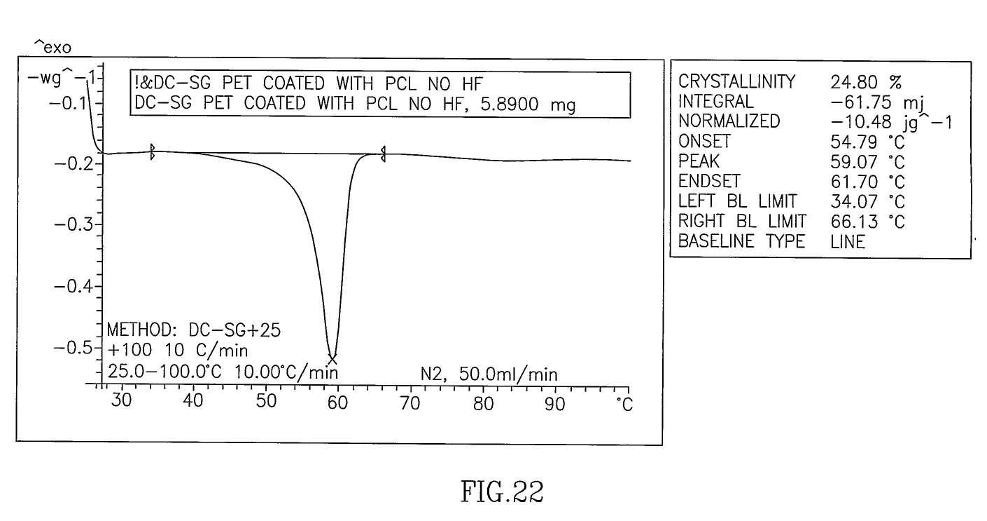

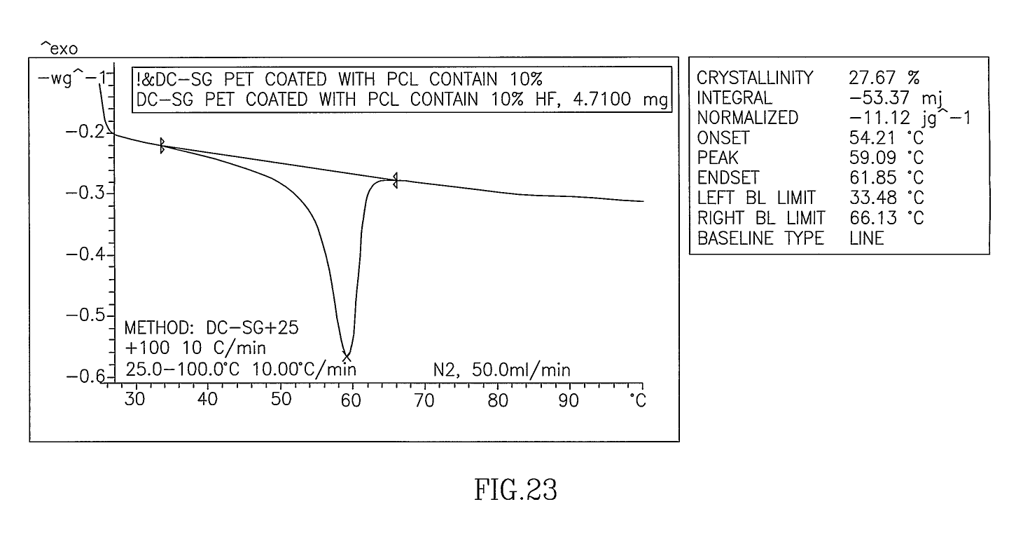

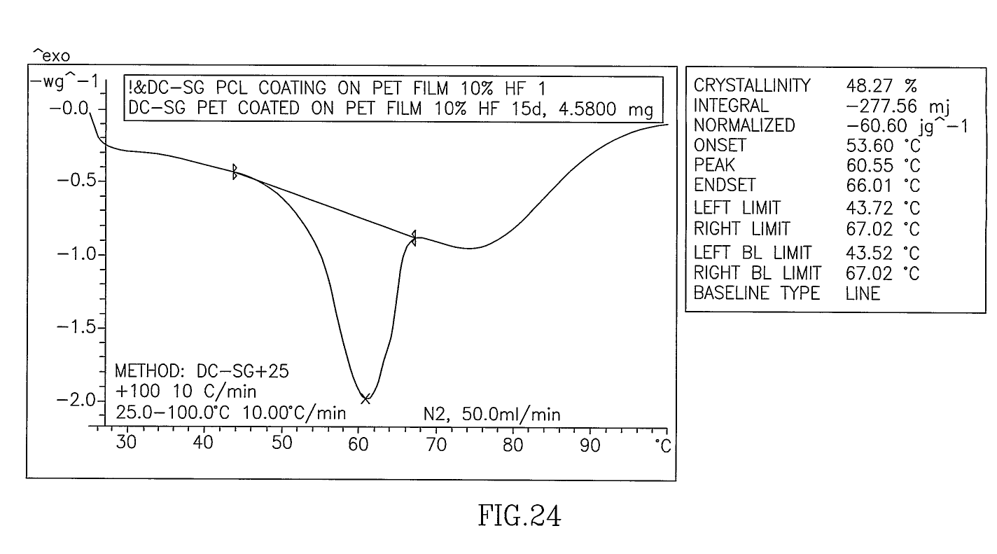

This behavior can be attributed to the combined effect of two factors. The first pertains to the much thinner PCL coatings used in this case (around 70 μm), compared to the PCL films described previously (around 200 μm). Clearly, the thinner the film, the higher the percentage of drug presents on the surface and, consequently, the more significant is the burst effect. The second factor has to do with the morphology of the PCL matrix. While the slow evaporation of the solvent used in the preparation of the PCL films resulted in a significantly crystalline material (64% degree of crystallinity, as reported in Table 4), the dipping and fast solvent evaporation technique generated rather amorphous PCL thin coatings. As shown in FIGS. 22-24 and Table 5, the degree of crystallinity of the coating was markedly low (around 25%). The limited degree of crystallinity of the PCL coating resulted also in a somewhat faster rate of degradation.

TABLE 5

| Sample | PCL crystallinity (%) |
|---|---|
| PET film coated with PCL | 25 |
| PET film coated with PCL + 10% HF | 28 |
| PET film coated with PCL + 10% HF | 48 |
| 15 days in PBS | |

In another system, stainless steel bars were coated by dipping them in a PCL 10% w/w THF solution. After dipping for 2 minutes, the solvent was evaporated and 50 to 100 μm thick coating layers were formed, with an average weight increase of approximately 2% (as related to the metal). Halofuginone-containing PCL coatings were prepared by dipping the metal bars into a dispersion of the drug in THF, having 5 and 10% w/w drug loadings.

### Example 4

### Polymeric Coating of Halofuginone Particles

The following experiments were conducted with solid drug particles coated with PCL. Here, 4%-7% HF was stirred in a 0.1% w/w PCL solution in THF, followed by the evaporation of the solvent under constant stirring in a rotovapor. Initially, work had to be devoted to determining the appropriate PCL concentration, to prevent or minimize the formation of PCL films, as opposed to coating the solid drug.

### Example 5

### Phase I Clinical Study to Determine the Safety of Halofuginone Administered Orally, using Three Different Dosing Regimens in Healthy Male Volunteers

The following results demonstrate the maximum tolerable dose of halofuginone, administered orally to human subjects. The results show that administering the maximum daily tolerable dose of halofuginone by multiple low doses reduces the side effects of the drug.

The Phase I clinical study described below was conducted in the PPD Development Clinical Pharmacology Unit, 72 Hospital Close, Evington, Leicester, United Kingdom, between September 2001 and October 2001. The objective of this study was to compare the safety and tolerability of halofuginone when a daily dose of 2 mg is administered using three different dosing regimens.

Methodology:

A single-center, open label, three-period, phase I study was performed. Eight subjects attended the PPD Development Clinic for pre-study screening during 3 weeks of dosing. Visits at the Clinic were as follows:

Period 1:

Subjects were admitted to the Clinic on the evening of Day-1. After an overnight fast, subjects received an oral dose of 0.25 mg halofuginone with a standard snack. Each subject received a total of eight doses at 3-hour intervals starting at approximately 10:00 h on Day 1, and remained resident in the Clinic until the morning of Day 3 [after the 24 hour pharmacokinetic (PK) sample]. They returned to the Clinic for an out-patient visit on the morning of Day 4 to provide a PK blood sample. There was a washout of at least 6 days after the last dose in Period 1 before the start of dosing in Period 2.

Period 2:

Subjects were admitted to the Clinic at approximately 07:00 h on Day 1. Subjects were provided with breakfast and then fasted until the first dose. Subjects received an oral dose of 0.5 mg halofuginone with a standard meal at 6-hour intervals starting at approximately 13:00 h (a total of four doses) and remained resident in the Clinic until the morning of Day 3 (after the 24 hour PK sample). They returned to the Clinic for an out-patient visit on the morning of Day 4 to provide a PK blood sample. There was a washout of at least 6 days after the last dose in Period 2 before the start of dosing in Period 3.

Period 3:

Subjects were admitted to the Clinic on the evening of Day-1. After an overnight fast, subjects received a single oral dose of 2 mg halofuginone with breakfast at approximately 07:00 h on Day 1 and remained resident in the Clinic until the morning of Day 2 (after the 24 hour PK sample). They returned to the clinic for an out-patient visit on the morning of Day 3 to provide a PK blood sample.

Safety:

Adverse events (AEs) were monitored throughout the study period. Vital signs (including blood pressure, pulse rate and oral body temperature), electrocardiogram (ECG), physical examinations and routine laboratory safety analyses were conducted at the pre-study screening and at the post-study follow-up visits.

Study subjects:

Eight healthy male Caucasian subjects were enrolled into the study. The mean age was 31.1 years (range 19-39 years), mean height was 176.6 cm (range 169-184 cm), mean weight was 78.9 kg (range 62.7-91.7 kg) and mean BMI was 25.2 kg/m² (range 21.9-27.9 kg/m²). All subjects completed the study.

Results:

Adverse Events (AE)s:

There were no serious AEs during the study. A total of 29 AEs were reported by seven subjects. One AE was reported pre-dose. Of the remaining 28 treatment-emergent AEs, 26 were considered to be mild and two were considered to be moderate in severity. One treatment-emergent AE was considered to be not related to the test product, two were considered unlikely to be related, seven were considered to be possibly related, seven were considered probably related and 11 AEs were considered to be definitely related to the test product. Five treatment-emergent AEs resolved with treatment and 23 resolved without treatment. The most commonly reported AEs were nausea (13 AEs), vomiting (6 AEs) and headache (3 AEs). Feeling hot was reported on two occasions. All other AEs were reported only once.

Period 1 (8×0.25 mg Halofuginone):

The treatment-related AEs reported during Period 1 are summarized in Table 6.

TABLE 6

| | | | | No. of | |
|---|---|---|---|---|---|
| | | | Onset time | Doses | |
| | | Relation- | relative to | Taken | |
| Adverse | | ship to | previous dose | before | |
| event | Severity | study drug | (hours:mins.) | event | Outcome |
| Feeling | Mild | Possible | 00:45 | 1 | Resolved |
| hot | | | | | without |
| | | | | | treatment |
| Feeling | Mild | Possible | 02:55 | 6 | Resolved |
| hot | | | | | without |
| | | | | | treatment |
| Nausea | Mild | Possible | 00:00 | 7 | Resolved |
| | | | | | without |
| | | | | | treatment |
| Earache | Mild | Unlikely | 46:30 | 8 | Resolved |
| | | | | | without |
| | | | | | treatment |
| Thrombo- | Mild | Not | 58:52 | 8 | Resolved |
| phlebitis | | related | | | without |
| | | | | | treatment |
| Loose | Mild | Possible | Not known | 8 | Resolved |
| pale | | | (more than | | without |
| stools | | | 3 days) | | treatment |

Six treatment-related AEs were reported by four subjects. Four AEs were considered possibly related to the test product: feeling hot (two AEs in one subject), nausea and loose pale stools. All AEs were considered to be mild in severity, and all AEs resolved without treatment.

Period 2 (4×0.5 mg Halofuginone):

The treatment-related AEs reported during Period 2 are summarized in Table 7.

TABLE 7

| | | | | Number | |
|---|---|---|---|---|---|
| | | | Onset time | of doses | |
| | | Relation- | relative to | taken | |
| Adverse | | ship to | previous dose | before | |
| event | Severity | study drug | (hours:mins.) | event | Outcome |
| Headache | Mild | Possible | 02:22 | 1 | Resolved |
| | | | | | without |
| | | | | | treatment |
| Nausea | Mild | Probable | 01:12 | 4 | Resolved |
| | | | | | without |
| | | | | | treatment |
| Vomited | Mild | Probable | 01:39 | 4 | Resolved |
| | | | | | without |
| | | | | | treatment |
| Nausea | Mild | Probable | 00:42 | 2 | Resolved |
| | | | | | without |
| | | | | | treatment |
| Nausea | Mild | Probable | 00:53 | 2 | Resolved |
| | | | | | without |
| | | | | | treatment |
| Nausea | Mild | Probable | 00:28 | 3 | Resolved |
| | | | | | without |
| | | | | | treatment |
| Vomited | Mild | Probable | 00:49 | 4 | Resolved |
| | | | | | without |
| | | | | | treatment |
| Nausea | Mild | Probable | 00:51 | 4 | Resolved |
| | | | | | without |
| | | | | | treatment |
| Headache | Moderate | Possible | 27:44 | 4 | Resolved |
| | | | | | with |
| | | | | | treatment † |

† see text below

Nine treatment-related AEs were reported by three subjects. Two AEs were considered possibly related to the test product: headache (two AEs in two subjects). Seven AEs were considered to be probably related to the test product: nausea (five AEs in three subjects) and vomiting (two AEs in two subjects). Eight AEs were considered to be mild and one (headache) was considered to be moderate in severity. The moderate headache started more than 24 hours after the final dose and required the administration of 400 mg ibuprofen 9 hours later for resolution of the AE. All instances of nausea and vomiting resolved without treatment.

Period 3 (1×2 mg Halofuginone):

The treatment-related AEs reported after one dose during Period 3 are summarized in Table 8.

TABLE 8

| | | | Onset time | |
|---|---|---|---|---|
| | | | relative to | |
| Adverse | | Relationship | previous dose | |
| event | Severity | to study drug | (hours:mins.) | Outcome |
| Vomited | Mild | Definite | 00:59 | Resolved without |
| | | | | treatment |
| Nausea | Mild | Definite | 00:35 | Resolved without |
| | | | | treatment |
| Nausea | Mild | Definite | 00:58 | Resolved with |
| | | | | treatment † |
| Nausea | Mild | Definite | 01:20 | Resolved without |
| | | | | treatment |
| Vomited | Mild | Definite | 01:38 | Resolved without |
| | | | | treatment |
| Vomited | Mild | Definite | 00:51 | Resolved without |
| | | | | treatment |
| Nausea | Mild | Definite | 00:33 | Resolved without |
| | | | | treatment |
| Nausea | Mild | Definite | 01:23 | Resolved without |
| | | | | treatment |
| Vomited | Mild | Definite | 01:48 | Resolved without |
| | | | | treatment |
| Coryza | Mild | Unlikely | 18:44 | Resolved with |
| | | | | treatment † |
| Headache | Moderate | Possible | 27:44 | Resolved with |
| | | | | treatment † |
| Nausea | Mild | Definite | 00:48 | Resolved without |
| | | | | treatment |
| Nausea | Mild | Definite | 01:16 | Resolved without |
| | | | | treatment |
| Vomited | Mild | Definite | 01:17 | Resolved without |
| | | | | treatment |

† see text below

Fourteen treatment-related AEs were reported by seven subjects. One AE (coryza) was considered unlikely to be related to the test product and one AE (headache) was considered to be possibly related. Twelve AEs were considered to be definitely related to the test product: nausea (seven AEs in seven subjects), vomiting (five AEs in five subjects). Thirteen AEs were considered to be mild and one (headache) was considered to be moderate in severity. The moderate headache started more than 24 hours after administration of the test product and required 1 g paracetamol 5½ hours later for resolution of the AE. The coryza started about 18 hours after administration of the test product and was then treated with paracetamol over several days. All instances of vomiting and 6 of the 7 instances of nausea resolved without treatment. One instance of nausea started about 1 hour after administration of the test product and required 10 mg intramuscular Maxalon (metoclopramide) about 1 hour later for resolution of the AE.

Summary:

Six AEs were reported in Period 1, nine in Period 2 and 14 in Period 3. Overall, the lowest number of AEs was seen in Period 1 when the daily dose of 2 mg halofuginone was split into 8 separate doses (0.25 mg each) separated by 3-hourly intervals. The incidence of AEs was highest in Period 3 where subjects were given a single 2 mg dose of halofuginone.

The most common AEs were nausea (13 AEs) and vomiting (seven AEs). Both AEs have been reported before in subjects who have received halofuginone and were not unexpected. However, no anti-emetics were administered prophylactically during the study. All AEs of nausea and vomiting were considered to be mild in severity. Most nausea and vomiting adverse events were short in duration. All vomiting AEs and most nausea AEs resolved within 1 hour and 43 minutes. Only two nausea AEs lasted longer than 1 hour and 43 minutes: (one for 10 hours and another for 3 hours and 32 minutes). All AEs resolved. Only one nausea AE required treatment (metoclopramide) for resolution. All other nausea and vomiting AEs resolved without treatment.

Safety Conclusions:

In conclusion, the results of the study show that 2 mg halofuginone is safe and well tolerated in healthy male subjects when given as eight doses of 0.25 mg with a snack or 4 doses of 0.5 mg with a meal. However, a single dose of 2 mg halofuginone with a meal is less well tolerated and provides some adverse effects such as vomiting and Nausea. Overall, a split-dose regimen of halofuginone did reduce the incidence of vomiting.

### Example 6

### Phase I Clinical Study to Determine the Pharmacokinetic Parameters of Halofuginone Administered Orally in Patients with a Solid Progressive Tumor

The present interim pharmacokinetic analysis was carried out by ASTER. Patients included were treated for solid tumour. On study Day 1 each patient received one oral dose of halofuginone, either 1 mg (one patient) or 2 mg (6 patients), and blood samples for pharmacokinetics were collected up to 72 hours (3 days) after dosing. Immediately after the collection of the 72 hour blood sample, patients started the multiple dose regimen consisting in morning daily dose administration of 1 mg (for subject No.1) or 2 mg (6 remaining patients) until study Day 15. Urine samples were collected over 48 hours after first dosing. Urine concentrations were only available for patients No.1, No.2, No.3 and No.4, and thus only 4 patients were included in the analysis of urine excretion data.

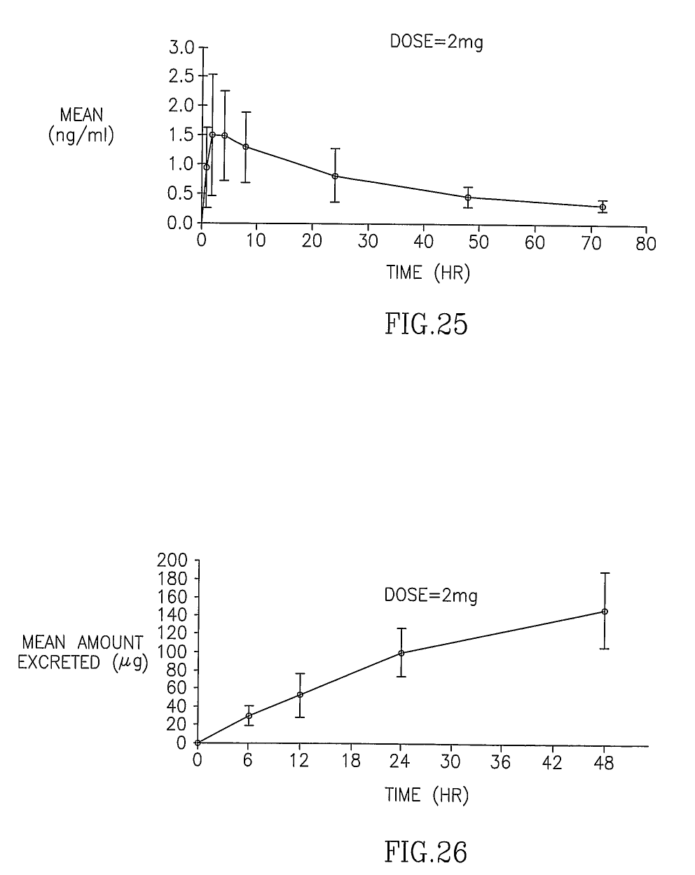

The mean and SD plasma concentration profiles of halofuginone obtained after treatment of 6 patients with single oral dose of 2 mg of halofuginone are presented in FIG. 25. After administration of a single oral dose of 2 mg halofuginone, the mean plasma concentration of halofuginone reached its maximum value (about 1.7 ng/mL) within 3 hours after dosing. Concentration of halofuginone then declined with a mean terminal half life of 37.2 hours.

The amount of halofuginone excreted to the urine was examined in 3 patients who were administered with a single oral dose of 2 mg halofuginone. The results presented in FIG. 26 revealed that in average the maximum amount of halofuginone excreted to the urine was 140 μg.

While the present invention has been particularly described, persons skilled in the art will appreciate that many variations and modifications can be made. Therefore, the invention is not to be construed as restricted to the particularly described embodiments, rather the scope, spirit and concept of the invention will be more readily understood by reference to the claims which follow.

1. A polymeric delivery system for sustained release administration of a quinazolinone derivative of formula (I) 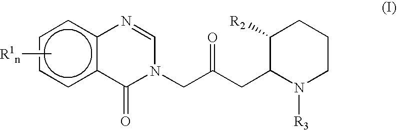
wherein: n=1-2
R₁ which may be the same or different at each occurrence is a member of the group consisting of hydrogen, halogen, nitro, benzo, lower alkyl, phenyl and lower alkoxy;
R₂ is a member of the group consisting of hydroxy, acetoxy and lower alkoxy;
R₃ is a member of the group consisting of hydrogen and lower alkenoxy-carbonyl; and pharmaceutically acceptable salts thereof, wherein the quinazolinone is released at a therapeutically effective dose for a period of at least one month.

2. The polymeric delivery system of claim 1 wherein the delivery system is formulated for local administration or topical administration to a target site in a subject.

3. The delivery system of claim 2, wherein the route of administration is selected from implantation, subcutaneous injection or deposition within a body cavity.

4. The delivery system of claim 1, wherein the quinazolinone derivative of formula (I) is halofuginone.

5. The delivery system of claim 3, wherein the delivery system is formulated as an implant, with the proviso that said implant does not comprise a stent.

6. A polymeric delivery system according to claim 1, comprising biocompatible two-phase polymeric beads comprising a core compartment, said core compartment comprising a water-in-oil emulsion surrounded by a polymeric shell compartment comprising a biocompatible polymer, wherein the discontinuous aqueous phase of the core compartment of the polymeric beads comprises the quinazolinone derivative of formula (I).

7. The delivery system of claim 6, wherein the biocompatible polymer is a natural or synthetic hydrophilic polymer.

8. The delivery system of claim 7, wherein the biocompatible hydrophilic polymer is a polysaccharide or a protein.

9. The delivery system of claim 8, wherein the polysaccharide is selected from the group consisting of: alginate, dextran, cellulose, cellulose derivatives, dextran sulfate, chondroitin sulfate, heparan sulfate, heparin, keratan sulfate, dermatan sulfate, and algal polyglycan sulfates.

10. (canceled)

11. The delivery system of claim 8, wherein the protein is selected from the group consisting of: gelatin, collagen, elastin, fibrin and albumin.

12. (canceled)

13. The delivery system of claim 6, wherein the quinazolinone derivative of formula (I) is halofuginone.

14. The delivery system of claim 6, wherein the quinazolinone derivative of formula (I) is released at a therapeutically effective concentration for a time period ranging from several days to several months.

15. The delivery system of claim 6, wherein the delivery system is formulated for local administration or topical administration to a target site.

16. The delivery system of claim 15, wherein the route of administration is selected from the group consisting of implantation, subcutaneous injection or deposition within a body cavity.

17. The delivery system of claim 6, wherein the polymeric beads are dispersed within an oil-based formulation or water-based selected from the group consisting of an oily suspension, emulsion, cream and gel.

18. A polymeric delivery system according to claim 1 comprising a biocompatible polymeric film wherein the quinazolinone derivative of formula (I) is homogeneously dispersed within the film.

19. The delivery system of claim 18, wherein the biocompatible polymer is a selected from the group consisting of a synthetic biodegradable and a synthetic non-biodegradable polymer.

20. The delivery system of claim 19, wherein the synthetic polymer is selected from: polyacrylic acid polymers, polylactic acid polymers, polycaprolactone polymers, polyglycolic acid and copolymers thereof.

21. (canceled)

22. The delivery system of claim 18, wherein the polymeric film is a coating of an article.

23. The delivery system of claim 18, wherein the quinazolinone derivative of formula (I) is halofuginone.

24. The delivery system of claim 18, wherein the quinazolinone derivative of formula (I) is released at a therapeutically effective concentration for a time period ranging from several days to several months.

25. The delivery system of claim 18, wherein the delivery system is suitable adapted for a route of administration selected from subcutaneous implantation and deposition within a body cavity.

26. The delivery system of claim 18, wherein the delivery system is adapted for application topically at a target site of a subject.

27. A polymeric delivery system according to claim 1 comprising a polymeric complex comprising at least one type of biocompatible negatively-charged polymeric molecule conjugated through electrostatic interactions to the quinazolinone derivative of formula (I), said quinazolinone derivative of formula (I) having a positive charge at physiological pH.

28. The delivery system of claim 27, wherein the negatively charged biocompatible polymer comprises a synthetic or natural biocompatible polymer.

29. The delivery system of claim 28, wherein the synthetic or natural polymer is selected from the group consisting of polyacrylic acid polymers, alginate polymers, polylactic acid polymers, polyglycolic acid and copolymers thereof.

30. (canceled)

31. The delivery system of claim 27, wherein the quinazolinone derivative of formula (I) is halofuginone.

32. The delivery system of claim 27, wherein the quinazolinone derivative of formula (I) is released at a therapeutically effective concentration for a time period ranging from several days to several months.

33. The delivery system of claim 27, wherein the delivery system is adapted for a route of administration selected from subcutaneous implantation and deposition within a body cavity.

34. The delivery system of claim 27, wherein the delivery system is adapted for application topically at a target site of a subject.

35. A polymeric delivery system according to claim 1 comprising biocompatible polymeric beads in suspension, wherein the polymeric beads comprise the quinazolinone derivative of formula (I).

36. The delivery system of claim 35, wherein the biocompatible polymer is a natural or synthetic hydrophilic polymer.

37. The delivery system of claim 36, wherein the biocompatible natural polymer is selected from the group consisting of a polysaccharide and a protein.

38. The delivery system of claim 37, wherein the polysaccharide is selected from the group consisting of: alginate, dextran, cellulose, cellulose derivatives, dextran sulfate, chondroitin sulfate, heparan sulfate, heparin, keratan sulfate, dermatan sulfate, and algal polyglycan sulfates.

39. (canceled)

40. The delivery system of claim 37, wherein the protein is selected from the group consisting of: gelatin, collagen, elastin, fibrin and albumin.

41. (canceled)

42. The delivery system of claim 35, wherein the quinazolinone derivative of formula (I) is halofuginone.

43. The delivery system of claim 35, wherein the quinazolinone derivative of formula (I) is released at a therapeutically effective concentration for a time period ranging from several days to several months.

44. The delivery system of claim 35, wherein the delivery system is adapted for a route of administration selected from the group consisting of implantation, subcutaneous injection and deposition within a body cavity.

45. The delivery system of claim 35, wherein the delivery system is formulated for topical administration to a target site in a subject.

46. The delivery system of claim 35, wherein the polymeric beads are dispersed within an oil-based or water-based formulation selected from the group consisting of an oily suspension, emulsion, cream or gel.

47. A method of preparing the biocompatible polymeric beads of claim 6 comprising:
mixing an aqueous suspension of the quinazolinone derivative of formula (I) in an oily phase to form a water-in-oil emulsion;
homogenizing said emulsion;
applying a polymeric shell around small droplets of the emulsion by core/shell extrusion, and
solidifying the shell to form two phase core-and-shell-structured polymeric beads.

48. A method of preparing the polymeric film of claim 18 comprising:
dissolving the quinazolinone derivative of formula (I) in an organic solvent to form a drug solution;
mixing the polymer in a solvent to form a polymeric solution;
mixing the drug solution with the polymeric solution; and
evaporating the polymer solvent to form the polymeric films comprising said quinazolinone derivative of formula (I) homogenously dispersed therein.

49. A method of preparing the biocompatible delivery system of claim 27 comprising:
dissolving the quinazolinone derivative of formula (I) in an aqueous phase to form a drug solution;
mixing the polymer in an aqueous phase to form a polymeric solution;
mixing the drug solution with the polymeric solution for sufficient time to form polymeric complexes; and
precipitating the polymeric complexes.

50. A method of preparing the biocompatible delivery system of claim 35 comprising:
suspending the quinazolinone derivative of formula (I) in an aqueous solution to form a drug suspension;
mixing the polymer in a solvent to form a polymeric solution;
mixing the polymeric solution with a cross linking agent and the drug suspension to form polymeric beads comprising said quinazolinone derivative of formula (I).

51-66. (canceled)

67. An implant comprising the polymeric delivery system of claim 6.

68. An implant comprising the polymeric delivery system of claim 18.

69. An implant comprising the polymeric delivery system of claim 27.

70. An implant comprising the polymeric delivery system of claim 35.
